(19) United States
(12) Patent Application Publication
(10) Pub. No.: US 2010/0019141 A1
(43) Pub. Date: Jan. 28, 2010

(54) ENERGY CONTAMINATION MONITOR WITH NEUTRAL CURRENT DETECTION

(75) Inventors: Joseph C. Olson, Beverly, MA (US); Robert L. Badzey, Quincy, MA (US)

Correspondence Address: VARIAN SEMICONDUCTOR EQUIPMENT ASSC., INC., 35 DORY RD., GLOUCESTER, MA, 01930-2297, US

(73) Assignee: VARIAN SEMICONDUCTOR EQUIPMENT ASSOCIATES, INC., Gloucester, MA (US)

(21) Appl. No.: 12/507,345

(22) Filed: Jul. 22, 2009

Related U.S. Application Data
(60) Provisional application No. 61/083,533, filed on Jul. 25, 2008.

(51) Int. Cl. B01D 59/44; H01J 27/00; H01J 49/00; G01K 1/08; A61N 5/00

(52) U.S. Cl. 250/282; 250/423 R; 250/426; 250/287; 250/397; 250/492.3

(57) ABSTRACT

This energy contamination monitor has an ionization apparatus configured to ionize the neutral particles in an ion beam. Neutral particles are ionized, separated based at least in part upon different transit times over a distance, and measured with the Faraday electrode based at least in part upon the different transit times. The energy contamination monitor can distinguish between fast and slow neutral particles.

### CROSS-REFERENCE TO RELATED APPLICATIONS

This application claims priority to the provisional patent application entitled “Energy Contamination Monitor with Neutral Current Detection” filed Jul. 25, 2008 and assigned U.S. Application No. 61/083,533, which is hereby incorporated by reference.

### FIELD

This invention relates to measuring energy contamination and, more particularly, to measuring energy contamination and detecting neutral particles.

### BACKGROUND

Ion implantation is a standard technique for introducing conductivity-altering impurities into semiconductor workpieces. A desired impurity material is ionized in an ion source, the ions are accelerated to form an ion beam of prescribed energy, and the ion beam is directed at the surface of the workpiece. The energetic ions in the beam penetrate into the bulk of the workpiece material and are embedded into the crystalline lattice of the workpiece material to form a region of desired conductivity.

An ion implanter includes an ion source for converting a gas or a solid material into a well-defined ion beam. The ion beam typically is mass analyzed to eliminate undesired ion species, accelerated to a desired energy, and implanted into a target. The ion beam may be distributed over the target area by electrostatic or magnetic beam scanning, by target movement, or by a combination of beam scanning and target movement. The ion beam may be a spot beam or a ribbon beam having a long dimension and a short dimension.

As the semiconductor industry reduces feature sizes on micro-electronic devices, ion beams with lower energies are desirable to achieve shallow implants in a workpiece. Yet these low energy ion beams de-neutralize and separate or “blow up” over a relatively short distance. It is thus desirable to obtain low-energy implants using a beam that has a short distance to travel between generation in an ion source and implantation in a workpiece. Unfortunately, in many instances one ion implanter is used to generate ions over multiple energies that may range from 1 keV to several hundred keV. Any high energy ions may need a long beamline for focusing and acceleration.

One method used to perform shallow implants is to generate a high energy ion beam that is decelerated at the end of the beamline to form a low energy ion beam for shallow implants. High energy beams may not suffer the separation or “blow up” effects to the extent low energy beams do. This method, however, is prone to energy contamination. Ions in the high energy ion beam may interact with each other or ambient gases to become neutral due to charge exchange before reaching the deceleration stage. This portion of the ion beam may not be properly decelerated due to its lack of charge, thus forming fast neutral particles. These fast neutral particles may have a kinetic energy that is approximately equivalent to the parent ion, which may be approximately 2 to 10 times greater than the ions used for implantation. Since these fast neutral particles are still in the ion beam when the ion beam impacts a workpiece, these may be implanted into the workpiece. Implantation of fast neutral particles causes problems in the workpiece because the fast neutral particles may be implanted deep into the exposed workpiece. The fast neutral particles also may cause problems with the parameters of the ion beam, such as dose, uniformity, or the dopant depth profile.

Slow neutral particles may be formed in the same manner as the fast neutral particles after the ion beam passes through a deceleration stage. Slow neutral particles also may cause problems in the workpiece if implanted and with parameters of the ion beam. Both slow and fast neutral particles may be difficult to detect because neither type of neutral particle has a net electric charge.

An ion beam is typically measured with a Faraday electrode. A Faraday electrode, however, cannot easily measure neutral particles. FIG. 1 is a cross-sectional view of an embodiment of an energy contamination monitor. The energy contamination monitor 100 has a first aperture 104, a pair of high-voltage electrodes 102, and a Faraday electrode 103. The ion beam 101, which may be a ribbon or spot beam, enters the energy contamination monitor 100 through the first aperture 104. This ion beam 101 contains both neutral particles and ions. The ion beam 101 impacts the Faraday electrode 103 and generates secondary electrons through the collisions with the surface of the Faraday electrode 103. By changing the bias of the high-voltage electrodes 102, different electron populations may be measured. For example, if a high positive voltage is applied to the high-voltage electrodes 102, ions within the ion beam 101 are substantially prevented from striking the Faraday electrode 103. Thus, mainly neutral particles of the ion beam 101 impact the Faraday electrode 103 and secondary electrons due to the neutral particles are measured. If a high negative voltage is applied to the high-voltage electrodes 102, secondary electrons are substantially prevented from being collected and mainly the ion current is measured. If no voltage or only a slightly positive voltage is applied to the high-voltage electrodes 102, secondary electrons from all atomic species in ion beam 101, both ionic and neutral, are measured.

There are shortcomings with this method of measuring neutral particles using a Faraday electrode. First, it is assumed that secondary electron generation is the same for both neutral and ionic species within the ion beam 101. Second, the energy contamination monitor 100 still cannot distinguish between fast and slow neutral particles. Accordingly, there is a need in the art for an apparatus and method to measure energy contamination that also can distinguish between fast and slow neutral particles within an ion beam.

### SUMMARY

According to a first aspect of the invention, an energy contamination monitor is provided. The energy contamination monitor comprises an ion beam having fast and slow neutral particles, an ionization apparatus configured to ionize the fast and slow neutral particles, and a Faraday electrode.

According to a second aspect of the invention, a method of measuring energy contamination in an ion beam is provided. The method comprises directing an ion beam having fast and slow neutral particles toward an entrance of an energy contamination monitor. The fast and slow neutral particles are ionized after the ion beam enters the energy contamination monitor through the entrance to form ionized fast and slow neutral particles. The ionized fast and slow neutral particles separate based at least upon different transit times of the ionized fast and slow neutral particles over a distance. The ionized fast and slow neutral particles are measured with a Faraday electrode based at least in part upon the different transit times.

According to a third aspect of the invention, a method of processing a workpiece in an ion implanter using a signal from an energy contamination monitor is provided. The method comprises directing an ion beam having fast and slow neutral particles toward an entrance of an energy contamination monitor. The fast and slow neutral particles are ionized after the ion beam enters the energy contamination monitor through the entrance to form ionized fast and slow neutral particles. The ionized fast and slow neutral particles separate based at least upon different transit times of the ionized fast and slow neutral particles over a distance. The ionized fast and slow neutral particles are measured with a Faraday electrode based at least in part upon the different transit times. A signal from the Faraday electrode is outputted and the ion beam is adjusted based upon the signal.

### BRIEF DESCRIPTION OF THE DRAWINGS

For a better understanding of the present disclosure, reference is made to the accompanying drawings, which are incorporated herein by reference and in which:

FIG. 1 is a cross-sectional view of an embodiment of an energy contamination monitor;

FIG. 2 is a block diagram of a beam-line ion implanter for implanting a material with ions;

FIG. 3 is a cross-sectional view of a second embodiment of an energy contamination monitor;

FIG. 4A is a block diagram of a possible location of the energy contamination monitor of FIG. 3;

FIG. 4B is a block diagram of another possible location of the energy contamination monitor of FIG. 3;

FIG. 4C is a block diagram of another possible location of the energy contamination monitor of FIG. 3; and

FIG. 5 is an example of Faraday electrode current when a signal is synchronized with the ionization apparatus.

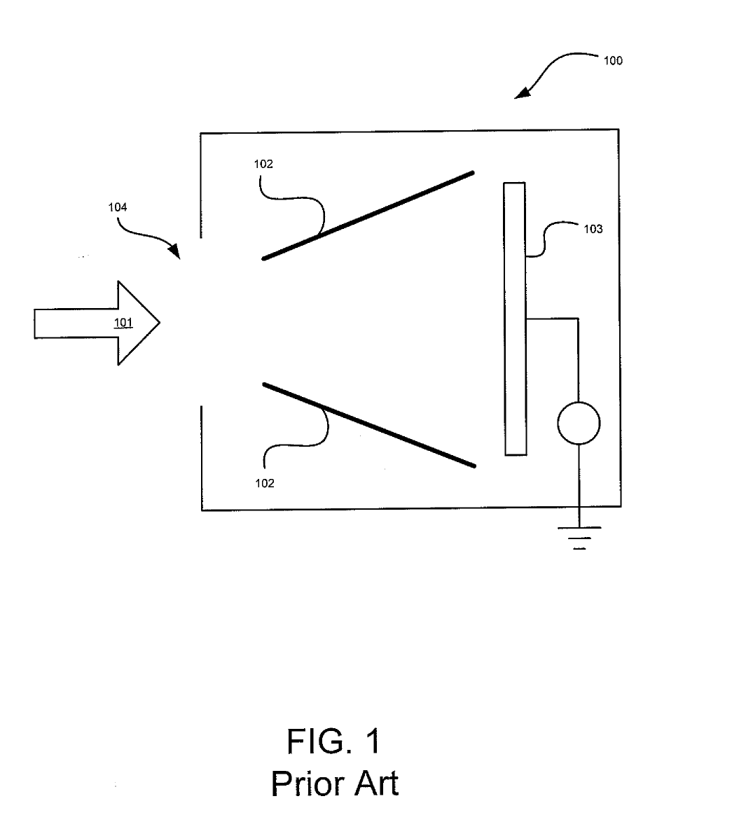

### DETAILED DESCRIPTION

The energy contamination monitor is described herein in connection with an ion implanter. However, the energy contamination monitor can be used with other systems and processes involved in semiconductor manufacturing or other systems that use ions, ion beams, or particle beams with neutral particles. Thus, the invention is not limited to the specific embodiments described below.

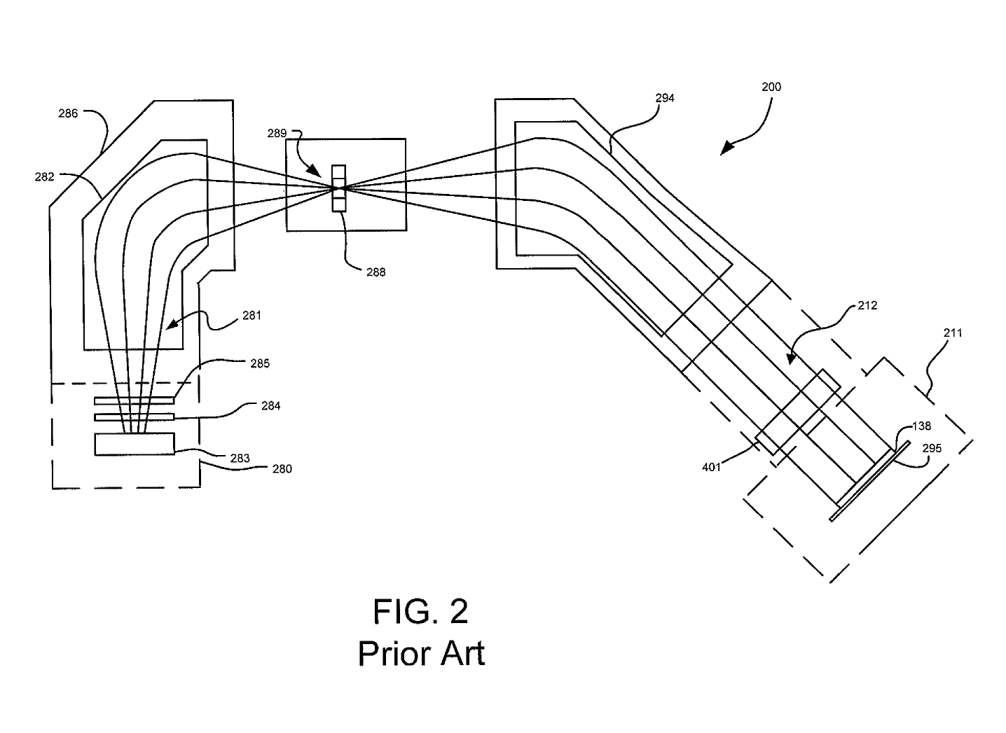

Turning to FIG. 2, a block diagram of a beam-line ion implanter 200 that may provide ions for implanting a selected material is illustrated. A person of ordinary skill in the art will recognize that the beam-line ion implanter 200 is only one of many examples of beam-line ion implanters that can provide ions for implanting a selected material.

In general, the beam-line ion implanter 200 includes an ion source 280 to generate ions that form an ion beam 281. The ion source 280 may include an ion chamber 283 and a gas box containing a gas to be ionized. The gas is supplied to the ion chamber 283 where the gas is ionized. The ions thus formed are extracted from the ion chamber 283 to form the ion beam 281. The ion beam 281 is directed between the poles of resolving magnet 282. A power supply is connected to an extraction electrode of the ion source 280 and provides an adjustable voltage.

The ion beam 281 passes through a suppression electrode 284 and ground electrode 285 to the mass analyzer 286. The mass analyzer 286 includes a resolving magnet 282 and a masking electrode 288 having a resolving aperture 289. Resolving magnet 282 deflects ions in the ion beam 281 such that ions of a desired ion species pass through the resolving aperture 289. Undesired ion species do not pass through the resolving aperture 289, but are blocked by the masking electrode 288.

Ions of the desired ion species pass through the resolving aperture 289 to the angle corrector magnet 294. The angle corrector magnet 294 deflects ions of the desired ion species and converts the ion beam from a diverging ion beam to a ribbon ion beam 212, which has substantially parallel ion trajectories. The beam-line ion implanter 200 may further include, for example, an electrostatic deceleration unit 401 upstream of the end station 211 in one embodiment. Other embodiments include an acceleration unit.

An end station 21 1 supports one or more workpieces, such as the workpiece 138, in the path of the ribbon ion beam 212 such that ions of the desired species are implanted into workpiece 138. In one instance, the workpiece 138 may be a semiconductor wafer having a disk shape, such as, in one embodiment, a 300 mm diameter silicon wafer. However, the workpiece 138 is not limited to a silicon wafer. The workpiece 138 could also be, for example, a flat panel, solar, or polymer substrate. The end station 211 may include a platen 295 to support the workpiece 138. The end station 211 also may include a scanner (not shown) for moving the workpiece 138 perpendicular to the long dimension of the ribbon ion beam 212 cross-section, thereby distributing ions over the entire surface of workpiece 138. Although the ribbon ion beam 212 is illustrated, other ion implanter embodiments may provide a spot beam.

The ion implanter may include additional components known to a person of ordinary skill in the art. For example, the end station 211 typically includes automated workpiece handling equipment for introducing workpieces into the beam-line ion implanter 200 and for removing workpieces after ion implantation. The end station 211 also may include a dose measuring system, an electron flood gun, or other known components. It will be understood to a person of ordinary skill in the art that the entire path traversed by the ion beam is evacuated during ion implantation. The beam-line ion implanter 200 may incorporate hot or cold implantation of ions in some embodiments.

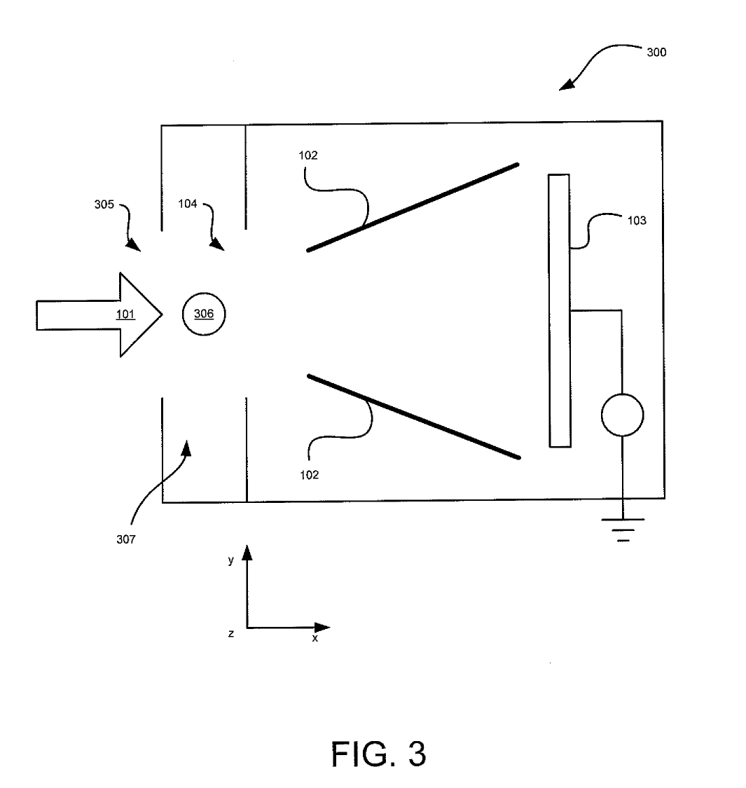

FIG. 3 is a cross-sectional view of a second embodiment of an energy contamination monitor. Energy contamination monitor 300 has a first aperture 104, a pair of high-voltage electrodes 102, and a Faraday electrode 103. In some embodiments, the pair of high-voltage electrodes 102 is replaced with a single high-voltage cylinder, a single electrode, or a magnet. The high-voltage electrodes 102 may be adjustably energized. In other embodiments, a micro-channel plate assembly, channeltron, or pyrometer/bolometer may be used instead of the Faraday electrode 103. Other measurement devices also may be used and, thus, the apparatus is not limited solely to the Faraday electrode 103. The energy contamination monitor 300 further has a second aperture 305 and an ionization beam 306.

The ion beam 101, which may be a ribbon or spot beam, enters the energy contamination monitor 300 through the second aperture 305. This ion beam 101 contains both neutral particles and ions. The ionization beam 306 will then ionize the ion beam 101 such that any neutral particles become charged. The ion beam 101 then passes through the first aperture 104 and impacts the Faraday electrode 103 to generate secondary electrons through the collisions with the surface of the Faraday electrode 103. The ionization of the neutral particles by the ionization beam 306 will allow for detection of energy contamination using time-of-flight.

The placement of the second aperture 305 lets the ion beam 101 enter an ionization region 307. In this particular embodiment, the ionization beam 306 is directed at the ion beam 101 in the ionization region 307 upstream or before the high-voltage electrodes 102. The ionization region 307 is an area where the neutral particles in the ion beam 101 may be ionized. The ionization region 307 is configured to minimize its impact on the rest of the energy contamination monitor 300. In yet another embodiment, the ionization beam 306 is directed at the ion beam 101 upstream of the entire energy contamination monitor 300.

The ionization beam 306 crosses the ion beam 101. This ionization beam 306 may be directed in any combination of the x, y, or z dimensions illustrated in FIG. 3. The ionization beam 306 may be an electron or photon beam. The ionization beam 306 also may be other ionization means.

In one particular embodiment, the ionization beam 306 is made up of collimated monoenergetic electrons and is aimed at a spot in the ionization region 307 between the first aperture 104 and second aperture 305. By changing the accelerating voltage of the electrons in the ionization beam 306, the number of ionized neutral particles can be maximized by achieving the maximum ionization cross-section. For typical atomic or molecular species, such as those including or composed of B, P, or As, this voltage may be between approximately 30 to 100 V. Different atomic species have different maximum cross-sections for electron impact ionization. This ionization lo apparatus that generates ionization beam 306 may be, for example, a hot filament, an electron cyclotron resonance (ECK) plasma source, or an indirectly-heated cathode (IHC). A hot filament is biased and heated to emission temperature to supply electrons for electron impact ionization. An ECR plasma source uses ECR to heat a plasma by injecting microwaves into a volume at a frequency that will heat free electrons, which then collide with atoms or molecules to cause ionization. An IHC has a filament that is disposed adjacent a cathode. This filament is heated and emits electrons that are propelled at the cathode. The cathode is heated and begins emitting electrons for electron impact ionization.

In another particular embodiment, the ionization beam 306 is composed of photons. Generally, photoionization and electron-impact ionization cross-sections and ionization energies are comparable. For photoionization, the ionization beam 306 will typically have a wavelength in the near-UV range of approximately 10 to 100 nm. Other wavelengths of light that ionize the neutral particles in the ion beam 101 also may be used. Photons may be configured to be in the energy range of maximal cross-section and to be well-focused on the ion beam 101. In one embodiment, the ionization apparatus that generates ionization beam 306 may be a photoionization source. This photoionization source may be a laser source or a monoenergetic arc-filament discharge source. UV sources, lights, lamps, or other photon-generating means also may be used.

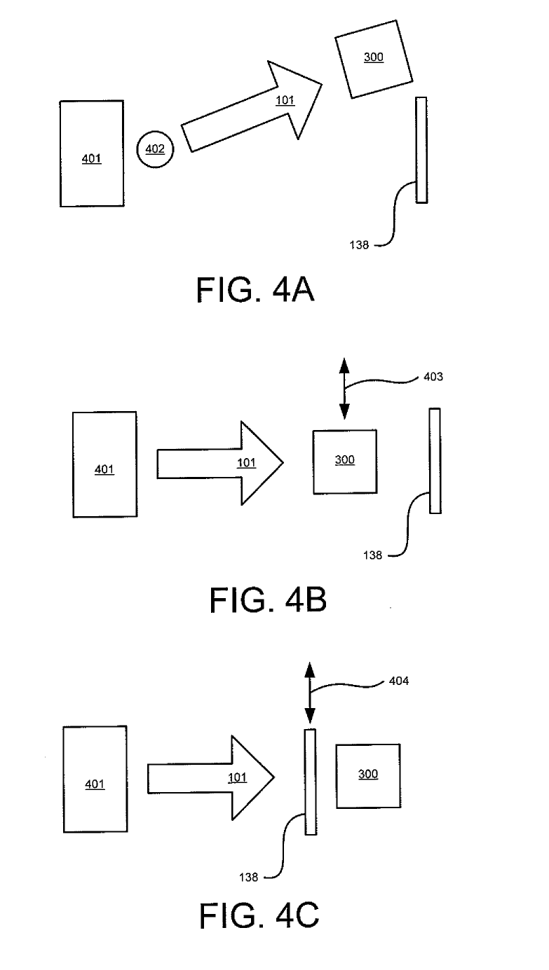

FIGS. 4A-4C are block diagrams of possible locations of the energy contamination monitor of FIG. 3. Depending on the embodiment, the energy contamination monitor 300 may be located in the end station 211 or in proximity to the workpiece 138. Thus, the energy contamination monitor 300 is placed downstream of any deceleration unit 401 and is configured to measure the energy contamination that would impact with the workpiece 138. The energy contamination monitor 300 also may be located elsewhere along the path of the ion beam to measure energy contamination. In these embodiments, the workpiece 138 may be removed while measuring energy contamination with the energy contamination monitor 300.

As seen in the embodiment of FIG. 4A, the ion beam 101 may be scanned or steered by scanner 402 toward the energy contamination monitor 300. The scanner 402 may be electrostatic or magnetic. The ion beam 101 may then be subsequently scanned or steered to implant the workpiece 138.

In the embodiment of FIG. 4B, the energy contamination monitor 300 moves in or out of the path of the ion beam 101 before the ion beam 101 implants the workpiece 138. This is illustrated by arrow 403. The ion beam 101 may then be subsequently implanted into the workpiece 138.

In the embodiment of FIG. 4C, the workpiece 138 may move in and out of the ion beam 101 and the energy contamination monitor 300 is located behind where the workpiece 138 would be located during implantation. This movement is illustrated by arrow 404. The ion beam 101 may then be subsequently implanted into the workpiece 138 after the workpiece 138 is placed in the path of the ion beam 101.

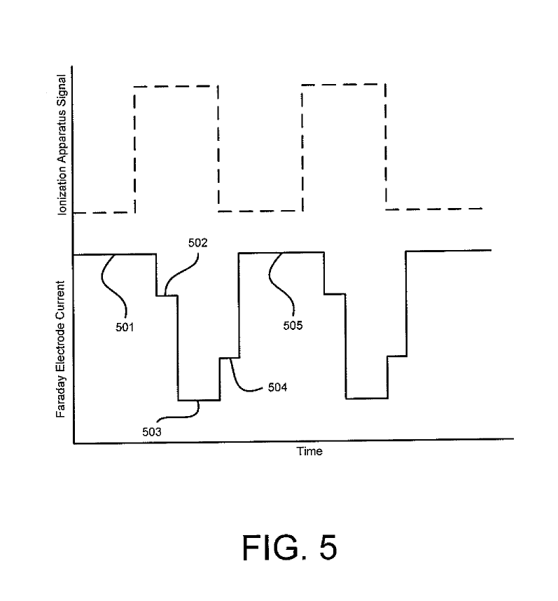

Measuring the neutral particles in the ion beam 101 and ionizing the neutral particles in the ion beam 101 are synchronized in some embodiments. By synchronizing the Faraday electrode 103 signal with the ionization apparatus that generates the ionization beam 306, changes in the current of the Faraday electrode 103 can be correlated with the ionized neutral particles in the ion beam 101. FIG. 5 is an example of Faraday electrode current when a signal is synchronized with the ionization apparatus. As the ionization apparatus changes parameters, the Faraday electrode 103 current changes.

While the ionization apparatus is off or operating in a low setting, the Faraday electrode current will be at a first point 501. At first point 501, all neutrals, both fast and slow, will be reaching the Faraday electrode 103, meaning that neutral particles in the ion beam 101 may move from the first aperture 104 to the Faraday electrode 103. Because the deceleration unit 401 had little or no effect on the fast neutral particles in the ion beam 101, these will move faster than the slow neutral particles or ions in the ion beam 101. Thus, the fast neutral particles in the ion beam 101 may separate from the slow neutral particles and ions in the ion beam 101 and may reach the Faraday electrode 103 first.

Prior to a second point 502, the ionization apparatus is turned on or begins operating at a higher setting. This generates or changes the parameters of the ionization beam 306. As illustrated in FIG. 5, there may be a delay between turning the ionization apparatus on or using a higher setting on the ionization apparatus and the second point 502 in the Faraday electrode current.

At a second point 502, the fast neutral particles in an ion beam 101 may be measured. Fast neutral particles in the ion beam 101 have a greater kinetic energy and velocity than ions or slow neutral particles in the ion beam 101 because deceleration did not substantially affect these fast neutral particles. Thus, these may have approximately the same energy and velocity as the ions in the ion beam 101 did before deceleration by the deceleration unit 401.

At a third point 503, the slow neutral particles and ions in an ion beam 101 may be measured. This is because the ions in an ion beam 101 that were neutralized after deceleration have approximately the same kinetic energy as the decelerated ions in the ion beam 101. Thus, these slow neutral particles and ions in the ion beam 101 will arrive at the Faraday electrode 103 at approximately the same time, but substantially after the fast neutral particles. The mass and energy of the fast and slow neutral particles also may contribute to velocity.

At the fourth point 504 in the Faraday electrode current, the ionization apparatus is turned off or begins operating again at a lower setting. The Faraday electrode current at the fourth point 504 will measure fast neutral particles in the beam that were ionized as the ionization apparatus was turned off or began operating at a lower setting. Some ionized slow neutral particles or ions also may be measured by the Faraday electrode 103. All neutral particles will then be measured by the Faraday electrode at fifth point 505. In one particular embodiment, this process may then be repeated as illustrated in FIG. 5.

These changes in the Faraday electrode current may differentiate fast and slow neutral particles. The distance between the first aperture 104 and the Faraday electrode 103 may be approximately 20 cm in some embodiments. The time separation between any fast and slow neutral particles may be measured in milliseconds, which may be differentiated with a controller. Thus, fast and slow neutral particles may be differentiated with this apparatus and method. The energy contamination monitor 300 may generate a signal representing these fast and slow neutral particles.

A controller, such as a controller in the beam-line ion implanter 200 or a controller for the energy contamination monitor 300, may then measure the fast and slow neutral particles through time-of-flight and differentiate between the fast and slow neutral particles. This controller may determine a value of the fast neutral particles and the slow neutral particles based at least in part upon their different transit times and may determine energy contamination levels in the ion beam 101. The controller may control the implant dose, part of the beam-line ion implanter 200, or another parameter based on the energy contamination levels. This controller can be or include a general-purpose computer or network of general-purpose computers that may be programmed to perform desired input/output functions. The controller also can include other electronic circuitry or components, such as application specific integrated circuits, other hardwired or programmable electronic devices, discrete element circuits, etc. The controller also may include communication devices, data storage devices, and software. In one instance, the controller may provide output signals to the beam-line ion implanter 200 or components of the beam-line ion implanter 200 and may receive input signals from the Faraday electrode 103 or the energy contamination monitor 300. A person of ordinary skill in the art will recognize that the controller may receive input signals from other components of the beam-line ion implanter 200. A user interface system also may be part of the controller and may include devices such as touch screens, keyboards, user pointing devices, displays, printers, etc. to allow a user to input commands and/or data and/or to monitor the beam-line ion implanter 200 via the controller. Energy contamination may be reduced or other beam characteristics may be optimized or changed using this feedback from the energy contamination monitor 300.

In one embodiment, the ion beam may be adjusted based on the signal from the energy contamination monitor 300. In another embodiment, the components of the beam-line ion implanter 200 are adjusted based on the signal from the energy contamination monitor 300. The ion beam or components of the beam-line ion implanter 200 may be adjusted to reduce energy contamination. In some embodiments generation of the ion beam is stopped, the ion beam is blocked, or the implantation is otherwise stopped when this signal is above a predetermined energy contamination level. Other tuning or adjusting applications in an ion implanter or other apparatus that uses ions also may be performed.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

What is claimed is:

1. An energy contamination monitor comprising:
an ion beam having fast and slow neutral particles;
an ionization apparatus configured to ionize said fast and slow neutral particles; and
a Faraday electrode.

2. The energy contamination monitor of claim 1, wherein said ionization apparatus is selected from the group consisting of a hot filament, an ECR plasma source, and an indirectly-heated cathode.

3. The energy contamination monitor of claim 1, wherein said ionization apparatus is a laser or a monoenergetic arc-filament discharge source.

4. The energy contamination monitor of claim 1, wherein said ionization apparatus is disposed upstream of said Faraday electrode.

5. The energy contamination monitor of claim 1, further comprising a high-voltage electrode that is configured to be biased.

6. The energy contamination monitor of claim 1, wherein said Faraday electrode is synchronized with said ionization apparatus.

7. The energy contamination monitor of claim 1, further comprising a controller, said controller configured to measure said fast and slow neutral particles through time-of-flight and differentiate between said fast and slow neutral particles.

8. A method of measuring energy contamination in an ion beam comprising:
directing an ion beam having fast and slow neutral particles toward an entrance of an energy contamination monitor;
ionizing said fast and slow neutral particles after said ion beam enters said energy contamination monitor through said entrance to form ionized fast and slow neutral particles;
separating said ionized fast and slow neutral particles based at least upon different transit times of said ionized fast and slow neutral particles over a distance; and
measuring said ionized fast and slow neutral particles with a Faraday electrode based at least in part upon said different transit times.

9. The method of claim 8, wherein said fast and slow neutral particles are ionized with electrons.

10. The method of claim 8, wherein said fast and slow neutral particles are ionized with photons.

11. The method of claim 8, further comprising the step of synchronizing measuring said ionized fast and slow neutral particles with said ionizing said fast and slow neutral particles.

12. The method of claim 11, further comprising the step of determining a value of said fast neutral particles and said slow neutral particles based at least in part upon said different transit times.

13. A method of processing a workpiece in an ion implanter using a signal from an energy contamination monitor comprising:
directing an ion beam having fast and slow neutral particles toward an entrance of an energy contamination monitor;
ionizing said fast and slow neutral particles after said ion beam enters said energy contamination monitor through said entrance to form ionized fast and slow neutral particles;
separating said ionized fast and slow neutral particles based at least upon different transit times of said ionized fast and slow neutral particles over a distance;
measuring said ionized fast and slow neutral particles with a Faraday electrode based at least in part upon said different transit times;
outputting a signal from said Faraday electrode; and
adjusting said ion beam based upon said signal.

14. The method of claim 13, wherein said fast and slow neutral particles are ionized with electrons.

15. The method of claim 13, wherein said fast and slow neutral particles are ionized with photons.

16. The method of claim 13, further comprising the step of synchronizing said measuring said ionized fast and slow neutral particles with said ionizing said fast and slow neutral particles.

17. The method of claim 16, further comprising the step of determining a value of said fast neutral particles and said slow neutral particles based at least in part upon said different transit times.

18. The method of claim 13, wherein said adjusting said ion beam based on said signal further comprises stopping implantation of said ion beam when said signal is above a predetermined level.

19. The method of claim 13, wherein said adjusting said ion beam based on said signal comprises reducing the generation of said fast and slow neutral particles in said ion beam.
